(19) United States
(12) Patent Application Publication
(10) Pub. No.: US 2024/0350266 A1
(43) Pub. Date: Oct. 24, 2024

(54) PROSTHETIC HEART VALVE IMPLANT APPARATUS

(71) Applicant: Medtronic, Inc., Minneapolis, MN (US)

(72) Inventor: Grant Alexander Menon, Santa Rosa, CA (US)

(21) Appl. No.: 18/582,929

(22) Filed: Feb. 21, 2024

Related U.S. Application Data
(60) Provisional application No. 63/460,963, filed on Apr. 21, 2023.

(51) Int. Cl. A61F 2/24

(52) U.S. Cl. CPC A61F 2/2436

(57) ABSTRACT

A transcatheter heart valve implant apparatus includes a valve prosthesis extending along a longitudinal axis. An inner shaft member extends along a shaft axis through the valve prosthesis between a proximal shaft end and a distal shaft end. The inner shaft member includes a first lumen extending between the proximal shaft end and a first exit opening at the distal shaft end. The inner shaft member includes a second lumen extending between the proximal shaft end and a second exit opening spaced from the distal shaft end. A first guidewire extends through the first lumen and the first exit opening. A first end of the first guidewire is at a first anchoring location. A second guidewire extends through the second lumen and the second exit opening. A second end of the second guidewire is at a second anchoring location. Methods of implanting a valve prosthesis are provided.

### CROSS-REFERENCE TO RELATED APPLICATIONS

This application claims the benefit of U.S. Provisional Application No. 63/460,963, filed Apr. 21, 2023, the entire content of which is incorporated herein by reference.

### FIELD

The present disclosure relates generally to a transcatheter heart valve implant apparatus and, more particularly, to a transcatheter heart valve implant apparatus configured to resist unintended movement during deployment of a heart valve prosthesis, thereby enabling controlled positioning of the heart valve prosthesis within a patient.

### BACKGROUND

It is known to provide a heart valve implant apparatus for implanting a heart valve prosthesis within a target site of the vasculature of a patient. The heart valve prosthesis can expand from a radially-collapsed position to a radially-expanded position. However, delivery of the heart valve prosthesis can be difficult because expansion of the heart valve prosthesis during deployment can cause unintended movement, resulting in less-controlled positioning within a patient.

### SUMMARY

The following presents a simplified summary of the disclosure to provide a basic understanding of some aspects described in the detailed description.

In aspects, a transcatheter heart valve implant apparatus comprises an annular valve prosthesis extending along a longitudinal axis between an inflow end of the valve prosthesis and an outflow end of the valve prosthesis. The transcatheter heart valve implant apparatus comprises an inner shaft member extending along a shaft axis through the valve prosthesis between a proximal shaft end and a distal shaft end. The inner shaft member comprises a first lumen extending between the proximal shaft end and a first exit opening at the distal shaft end. The inner shaft member further comprises a second lumen extending adjacent to the first lumen between the proximal shaft end and a second exit opening spaced a distance from the distal shaft end. A shaft wall is positioned between the first lumen and the second lumen. The transcatheter heart valve implant apparatus comprises a first guidewire extending through the first lumen and the first exit opening. A first end of the first guidewire is configured to be positioned at a first anchoring location. The transcatheter heart valve implant apparatus comprises a second guidewire extending through the second lumen and the second exit opening. A second end of the second guidewire is configured to be positioned at a second anchoring location simultaneously with the first guidewire positioned at the first anchoring location.

In aspects, the transcatheter heart valve implant apparatus comprises a tip attached to the distal shaft end. The tip comprises a tip lumen that is aligned with the first lumen such that the first guidewire extends through the tip lumen.

In aspects, the second exit opening is spaced apart from the tip.

In aspects, the first anchoring location comprises a first pulmonary artery branch and the second anchoring location comprises a second pulmonary artery branch.

In aspects, the transcatheter heart valve implant apparatus comprises an outer sheath circumferentially surrounding the valve prosthesis and the inner shaft member. The outer sheath is movable relative to the inner shaft member such that in a first delivery position, the second exit opening is within a sheath chamber of the outer sheath, and in a second delivery position, the second exit opening is outside of the sheath chamber.

In aspects, the inner shaft member is configured to be rotatable relative to the outer sheath such that in a first rotational position, the second exit opening faces a first location of the outer sheath, and in a second rotational position, the second exit opening faces a second location of the outer sheath.

In aspects, the first lumen comprises a first diameter that is within a range from about 0.5 millimeters to about 1.5 millimeters, and the second lumen comprises a second diameter that is within a range from about 0.5 millimeters to about 1.5 millimeters.

In aspects, the first lumen is substantially parallel to the second lumen.

In aspects, a transcatheter heart valve implant apparatus comprises an annular valve prosthesis extending along a longitudinal axis between an inflow end of the valve prosthesis and an outflow end of the valve prosthesis. The transcatheter heart valve implant apparatus comprises an inner shaft member extending along a shaft axis through the valve prosthesis between a proximal shaft end and a distal shaft end. The inner shaft member comprises a first lumen extending between a first entry opening at the proximal shaft end and a first exit opening at the distal shaft end. The inner shaft member comprises a second lumen extending adjacent to the first lumen between a second entry opening at the proximal shaft end and a second exit opening spaced a distance from the distal shaft end. A shaft wall is positioned between the first lumen and the second lumen. The transcatheter heart valve implant apparatus comprises a first guidewire extending through the first entry opening, the first lumen, and the first exit opening. A first end of the first guidewire is configured to be positioned in a first pulmonary artery branch. The transcatheter heart valve implant apparatus comprises a second guidewire extending through the second entry opening, the second lumen, and the second exit opening. A second end of the second guidewire is configured to be positioned in a second pulmonary artery branch simultaneously with the first guidewire positioned in the first pulmonary artery branch.

In aspects, the transcatheter heart valve implant apparatus comprises a guide member attached to the proximal shaft end of the inner shaft member.

In aspects, the guide member comprises a first passageway that is aligned with the first entry opening of the first lumen such that the first guidewire extends through the first passageway, the first entry opening, and the first lumen. The first passageway extends along a first guide axis. The guide member comprises a second passageway that is aligned with the second entry opening of the second lumen such that the second guidewire extends through the second passageway, the second entry opening, and the second lumen. The second passageway extends along a second guide axis.

In aspects, an angle between the first guide axis and the second guide axis is within a range from about 20 degrees to about 60 degrees.

In aspects, the transcatheter heart valve implant apparatus comprises a tip attached to the distal shaft end. The tip comprises a tip lumen that is aligned with the first lumen such that the first guidewire extends through the tip lumen.

In aspects, the second exit opening is spaced apart from the tip.

In aspects, methods of implanting a valve prosthesis comprise positioning a first guidewire within a first lumen of an inner shaft member. The first lumen extends between a proximal shaft end of the inner shaft member and a distal shaft end of the inner shaft member. Methods comprise positioning a second guidewire within a second lumen of the inner shaft member. The second lumen extends between the proximal shaft end and a second exit opening spaced a distance from the distal shaft end. Methods comprise guiding the first guidewire through a first exit opening at the distal shaft end such that a first guidewire end of the first guidewire is positioned in a first anchoring location. Methods comprise moving a transcatheter heart valve implant apparatus comprising the inner shaft member and the valve prosthesis along the first guidewire toward a treatment site within a body lumen. Methods comprise guiding the second guidewire through the second exit opening such that a second guidewire end of the second guidewire is positioned in a second anchoring location. Methods comprise deploying the valve prosthesis at the treatment site while the first guidewire end is positioned in the first anchoring location and the second guidewire end is positioned in the second anchoring location.

In aspects, during the moving of the transcatheter heart valve implant apparatus, an outer sheath circumferentially surrounds the valve prosthesis and the inner shaft member, and the second exit opening is within a sheath chamber of the outer sheath.

In aspects, prior to guiding the second guidewire through the second exit opening, methods comprise moving one or more of the outer sheath relative to the inner shaft member or the inner shaft member relative to the outer sheath such that the second exit opening is outside the sheath chamber.

In aspects, prior to guiding the second guidewire through the second exit opening, methods comprise rotating the inner shaft member relative to the outer sheath such that the second exit opening is oriented facing the second anchoring location.

In aspects, the rotating occurs while the second exit opening is outside the sheath chamber.

In aspects, during the moving of the transcatheter heart valve implant apparatus, the second guidewire end is located within the second lumen. The first anchoring location comprises a first pulmonary artery branch and the second anchoring location comprises a second pulmonary artery branch.

Additional features and advantages of the aspects disclosed herein will be set forth in the detailed description that follows, and in part will be clear to those skilled in the art from that description or recognized by practicing the aspects described herein, including the detailed description which follows, the claims, as well as the appended drawings. It is to be understood that both the foregoing general description and the following detailed description present aspects intended to provide an overview or framework for understanding the nature and character of the aspects disclosed herein. The accompanying drawings are included to provide further understanding and are incorporated into and constitute a part of this specification. The drawings illustrate various aspects of the disclosure, and together with the description explain the principles and operations thereof.

### BRIEF DESCRIPTION OF THE DRAWINGS

These and other features, aspects and advantages are better understood when the following detailed description is read with reference to the accompanying drawings, in which:

FIG. 1 schematically illustrates example aspects of a transcatheter heart valve implant apparatus in accordance with aspects of the disclosure;

FIG. 2 schematically illustrates a distal end of the transcatheter heart valve implant apparatus in accordance with aspects of the disclosure;

FIG. 3 illustrates deployment of a heart valve prosthesis in accordance with aspects of the disclosure;

FIG. 4 illustrates deployment of the heart valve prosthesis in accordance with aspects of the disclosure;

FIG. 5 illustrates a cross-sectional view of the distal end of the transcatheter heart valve implant apparatus along lines 5-5 of FIG. 2 in accordance with aspects of the disclosure;

FIG. 6 illustrates a first guidewire extending through an inner shaft member of the transcatheter heart valve implant apparatus in accordance with aspects of the disclosure;

FIG. 7 illustrates a second guidewire extending through the inner shaft member of the transcatheter heart valve implant apparatus in accordance with aspects of the disclosure;

FIG. 8 illustrates the second guidewire extending from the inner shaft member in accordance with aspects of the disclosure;

FIG. 9 illustrates the first guidewire and the second guidewire simultaneously extending from the inner shaft member during deployment of the heart valve prosthesis in accordance with aspects of the disclosure;

FIG. 10 illustrates a side view of the transcatheter heart valve implant apparatus with the first guidewire positioned in a right pulmonary artery in accordance with aspects of the disclosure;

FIG. 11 illustrates a side view of the transcatheter heart valve implant apparatus with the inner shaft member moving relative to an outer sheath in accordance with aspects of the disclosure;

FIG. 12 illustrates a side view of the inner shaft member rotating relative to the outer sheath in accordance with aspects of the disclosure;

FIG. 13 illustrates a side view of the transcatheter heart valve implant apparatus with the second guidewire positioned in a left pulmonary artery in accordance with aspects of the disclosure; and

FIG. 14 illustrates the first guidewire and the second guidewire simultaneously positioned in pulmonary arteries during deployment of the heart valve prosthesis in accordance with aspects of the disclosure.

### DETAILED DESCRIPTION

Aspects will now be described more fully hereinafter with reference to the accompanying drawings in which example aspects are shown. Whenever possible, the same reference numerals are used throughout the drawings to refer to the same or like parts. However, this disclosure may be embodied in many different forms and should not be construed as limited to the aspects set forth herein.

As used herein, the term “about” means that amounts, sizes, formulations, parameters, and other quantities and characteristics are not, and need not be, exact, but may be approximate and/or larger or smaller, as desired, reflecting tolerances, conversion factors, rounding off, measurement error and the like, and other factors known to those of skill in the art.

Ranges can be expressed herein as from “about” one value, and/or to “about” another value. When such a range is expressed, aspects include from the one value to the other value. Similarly, when values are expressed as approximations by use of the antecedent “about,” it will be understood that the value forms another aspect. It will be further understood that the endpoints of each of the ranges are significant both in relation to the other endpoint, and independently of the other endpoint.

Directional terms as used herein—for example up, down, right, left, front, back, top, bottom, upper, lower, etc.—are made only with reference to the figures as drawn and are not intended to imply absolute orientation.

Unless otherwise expressly stated, it is in no way intended that any methods set forth herein be construed as requiring that its steps be performed in a specific order, nor that with any apparatus, specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred in any respect. This holds for any possible non-express basis for interpretation, including matters of logic relative to arrangement of steps, operational flow, order of components, or orientation of components; plain meaning derived from grammatical organization or punctuation, and; the number or type of aspects described in the specification.

As used herein, the singular forms “a,” “an,” and “the” include plural references unless the context clearly dictates otherwise. Thus, for example, reference to “a” component includes aspects having two or more such components, unless the context clearly indicates otherwise.

The word “exemplary,” “example,” or various forms thereof are used herein to mean serving as an example, instance, or illustration. Any aspect or design described herein as “exemplary” or as an “example” should not be construed as preferred or advantageous over other aspects or designs. Furthermore, examples are provided solely for purposes of clarity and understanding and are not meant to limit or restrict the disclosed subject matter or relevant portions of this disclosure in any manner. It can be appreciated that a myriad of additional or alternate examples of varying scope could have been presented but have been omitted for purposes of brevity.

As used herein, the terms “comprising,” “including,” and variations thereof shall be construed as synonymous and open-ended, unless otherwise indicated. A list of elements following the transitional phrases comprising or including is a non-exclusive list, such that elements in addition to those specifically recited in the list may also be present.

The terms “substantial,” “substantially,” and variations thereof as used herein are intended to represent that a described feature is equal or approximately equal to a value or description. For example, a “substantially planar” surface is intended to denote a surface that is planar or approximately planar. Moreover, “substantially” is intended to denote that two values are equal or approximately equal. The term “substantially” may denote values within about 10% of each other, for example, within about 5% of each other, or within about 2% of each other.

Modifications may be made to the instant disclosure without departing from the scope or spirit of the claimed subject matter. Unless specified otherwise, “first,” “second,” or the like are not intended to imply a temporal aspect, a spatial aspect, an ordering, etc. Rather, such terms are merely used as identifiers, names, etc. for features, elements, items, etc. For example, a first end and a second end generally correspond to end A and end B or two different ends.

Unless otherwise indicated, the terms “distal” and “proximal” are used in the following description with respect to a position or direction relative to the treating clinician. “Distal” and “distally” are positions distant from or in a direction away from the clinician, and “proximal” and “proximally” are positions near or in a direction toward the clinician. In addition, the term “self-expanding” may be used in the following description with reference to one or more valve or stent structures of the prostheses hereof and is intended to convey that the structures are shaped or formed from a material that can be provided with a mechanical memory to return the structure from a compressed or constricted delivery configuration to an expanded deployed configuration or vice versa. Non-exhaustive exemplary self-expanding materials include stainless steel, a pseudo-clastic metal such as a nickel titanium alloy or nitinol, various polymers, or a so-called super alloy, which may have a base metal of nickel, cobalt, chromium, or other metal. Mechanical memory may be imparted to a wire or stent structure by thermal treatment to achieve a spring temper in stainless steel, for example, or to set a shape memory in a susceptible metal alloy, such as nitinol. Various polymers that can be made to have shape memory characteristics may also be suitable for use in aspects hereof to include polymers such as polynorborene, trans-polyisoprene, styrene-butadiene, and polyurethane. As well poly L-D lactic copolymer, oligo caprylactone copolymer and poly cyclo-octine can be used separately or in conjunction with other shape memory polymers.

Diseases associated with heart valves, such as those caused by damage or a defect, can include stenosis and valvular insufficiency or regurgitation. For example, valvular stenosis causes the valve to become narrowed and hardened which can prevent blood flow to a downstream heart chamber from occurring at the proper flow rate and may cause the heart to work harder to pump the blood through the diseased valve. Valvular insufficiency or regurgitation occurs when the valve does not close completely, allowing blood to flow backwards, thereby causing the heart to be less efficient. A diseased or damaged valve, which can be congenital, age-related, drug-induced, or in some instances, caused by infection, can result in an enlarged, thickened heart that loses elasticity and efficiency. Some symptoms of heart valve diseases can include weakness, shortness of breath, dizziness, fainting, palpitations, anemia and edema, and blood clots which can increase the likelihood of stroke or pulmonary embolism. Symptoms can often be severe enough to be debilitating and/or life threatening.

Heart valve prostheses have been developed for repair and replacement of diseased and/or damaged heart valves. Such heart valve prostheses can be percutaneously delivered and deployed at the site of the diseased heart valve through catheter-based delivery systems. Such heart valve prostheses generally include a frame or stent and a prosthetic valve mounted within the frame. Such heart valve prostheses are delivered in a radially compressed or crimped configuration so that the heart valve prosthesis can be advanced through the patient's vasculature. Once positioned at the treatment site, the heart valve prosthesis is expanded to engage tissue at the diseased heart valve region to, for instance, hold the heart valve prosthesis in position.

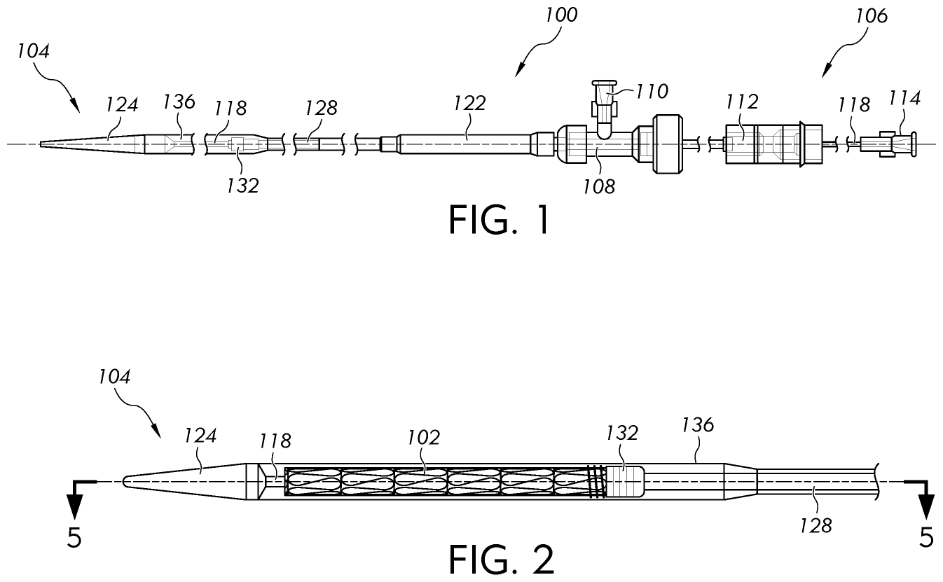

FIGS. 1-14 illustrate an example transcatheter heart valve implant apparatus 100 for delivering a heart valve prosthesis 102. The transcatheter heart valve implant apparatus 100 is illustrated to facilitate description of the disclosure. The following description of the transcatheter heart valve implant apparatus 100 and the heart valve prosthesis 102 is merely exemplary in nature and is not intended to limit the scope of the application.

FIG. 1 illustrates the transcatheter heart valve implant apparatus 100 having and extending between a distal end 104 and a proximal end 106. The distal end 104 can be used to load and deliver the heart valve prosthesis 102 (e.g., illustrated in FIG. 2). The proximal end 106 can comprise components, for example, those found in other catheter delivery systems for controlling transcatheter delivery of the heart valve prosthesis 102 through the vasculature of the patient and for controlling deployment of the heart valve prosthesis 102 at the target site. In aspects, the components at the proximal end 106 may comprise, for example, a first rotating homeostasis valve 108, a side access port 110, a second rotating homeostasis valve 112, and a guide member 114. The first rotating homeostasis valve 108 can comprise latex and can form a fluid seal to limit blood or other fluid from leaking out of the transcatheter heart valve implant apparatus 100 at the proximal end 106 or entry site into a patient. The side access port 110 is provided to inject contrast media or saline, for example, into the transcatheter heart valve implant apparatus 100. The second rotating homeostasis valve 112 may be similar to the first rotating homeostasis valve 108 and can limit blood or other fluid from leaking back through the transcatheter heart valve implant apparatus 100. In addition, the second rotating homeostasis valve 112 can allow wires, devices, and fluid to pass through to aid in the preparation, delivery, and deployment of the heart valve prosthesis 102. Furthermore, the second rotating homeostasis valve 112 can control the components of the distal end 104. For example, by manipulating (e.g., rotating) a portion of the second rotating homeostasis valve 112, a physician can control components of the distal end 104. The first and second homeostasis valves 108, 112 can be compatible with catheter-laboratory components and, unless otherwise noted, the heart valve implant apparatus 100 is not intended to be limited to the specific components and features disclosed.

In aspects, the transcatheter heart valve implant apparatus 100 can be inserted into a vessel or artery of a patient, for example, the femoral artery. The proximal end 106 can extend outside of the patient, for example, in the groin area, while the distal end 104 may be delivered intravascularly to an area at or near a pulmonary valve inside the body. However, insertion of the transcatheter heart valve implant apparatus 100 in other areas of the body are also contemplated. The transcatheter heart valve implant apparatus 100 can include one or more axial lumens to pass items (e.g., guidewires, valve prosthesis, contrast media, other catheters, etc.) through the transcatheter heart valve implant apparatus 100.

In aspects, the transcatheter heart valve implant apparatus 100 can comprise an inner shaft member 118 comprising at least one guidewire lumen. As used herein, a lumen comprises a cavity or hollow bore that extends through a structure, for example, the inner shaft member 118. The guide member 114 can be attached to the inner shaft member 118 at the proximal end 106. In aspects, the inner shaft member 118 can extend partially or completely along an entire length of the transcatheter heart valve implant apparatus 100 and may be slid or moved through other components of the transcatheter heart valve implant apparatus 100. As explained herein, a plurality of guidewires can extend through the inner shaft member 118 and can be used to guide the heart valve prosthesis 102 to a desired location. The transcatheter heart valve implant apparatus 100 can comprise an outer shaft 122 that is located between the distal end 104 and the proximal end 106, with the inner shaft member 118 extending through the outer shaft 122. The outer shaft 122 can limit blood from leaking around the transcatheter heart valve implant apparatus 100 and provides a relatively smooth, flexible length to enable the heart valve implant apparatus 100 to traverse through the vasculature of the patient.

In aspects, a tapered tip 124 can be attached to the inner shaft member 118 at the distal end 104. The tip 124 comprises a tapered shape to case the passage of the implant apparatus 100 into and through the vasculature. In aspects, a holding catheter 128 can be attached to the heart valve prosthesis 102. The holding catheter 128 can comprise a centrally located lumen surrounding the inner shaft member 118. The holding catheter 128 can slide relative to the inner shaft member 118 and may be controlled by the second rotating homeostasis valve 112 at the proximal end 106 of the implant apparatus 100. In aspects, a portion of the second rotating homeostasis valve 112 can be rotated or otherwise manipulated in order to rotate the holding catheter 128 or move the holding catheter 128 proximally and distally as desired. In aspects, a coil 132 can be attached to the distal end of the holding catheter 128. In this way, movement of the holding catheter 128, as controlled by the second rotating homeostasis valve 112, can control movement or rotation of the coil 132. The coil 132 can be removably attached to the valve prosthesis 102. As such, the coil 132 can allow for loading of the valve prosthesis 102, holding of the valve prosthesis 102 during delivery, and release of the valve prosthesis 102 upon reaching a treatment site. In aspects, rotation of the coil 132 can cause the valve prosthesis 102 to detach from the coil 132. The implant apparatus 100 can further comprise an outer sheath 136 that may comprise a low friction and flexible material, such as polytetrafluoroethylene (PTFE), polyurethane, silicone, or polyethylene. The outer sheath 136 can be sized and shaped to receive components near the distal end 104, for example, the inner shaft member 118, the coil 132, the valve prosthesis 102, etc.

FIG. 2 illustrates the distal end 104 of the implant apparatus 100. In aspects, the valve prosthesis 102 can be in a radially-compressed position during intraluminal delivery to a treatment site. In the radially-compressed position, the valve prosthesis 102 can be removably attached to the coil 132 and circumferentially surrounded by the outer sheath 136. The valve prosthesis 102 can extend coaxially with the inner shaft member 118, for example, with the inner shaft member 118 extending through a center of the valve prosthesis 102. The outer sheath 136 comprises a cross-sectional size (e.g., diameter) that is less than a cross-sectional size of the valve prosthesis 102 when the valve prosthesis 102 is in a radially-expanded position (e.g., illustrated in FIG. 4). As such, the outer sheath 136, along with the coil 132, can maintain the valve prosthesis 102 in the radially-compressed position during delivery.

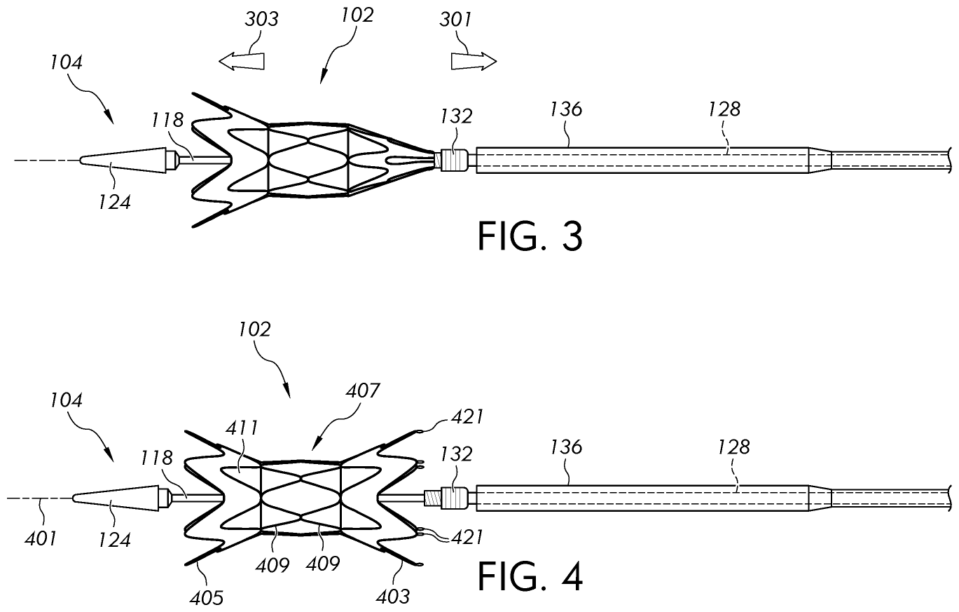

FIG. 3 illustrates initial stages of deployment of the valve prosthesis 102 upon reaching the treatment site (e.g., heart valve annulus, areas at or near a pulmonary valve, etc.). To deploy or release the valve prosthesis 102, the outer sheath 136 can be retracted, for example, by moving the outer sheath 136 relative to the valve prosthesis 102 in a proximal direction 301 away from the distal end 104 and toward the proximal end 106. In addition, or in the alternative, the holding catheter 128 can move relative to the outer sheath 136 in a distal direction 303 toward the distal end 104 and away from the proximal end 106, which can likewise cause the coil 132 and the valve prosthesis 102 to move in the distal direction 303. In this way, the valve prosthesis 102 may no longer be circumferentially surrounded by the outer sheath 136 and may begin to move from the radially-compressed position to the radially-expanded position. As illustrated in FIG. 3, an end of the valve prosthesis 102 may remain attached to the coil 132 while the valve prosthesis 102 is removed from the outer sheath 136 (e.g., due to the movement of the outer sheath 136 in the proximal direction 301 and/or movement of the holding catheter 128 in the distal direction 303).

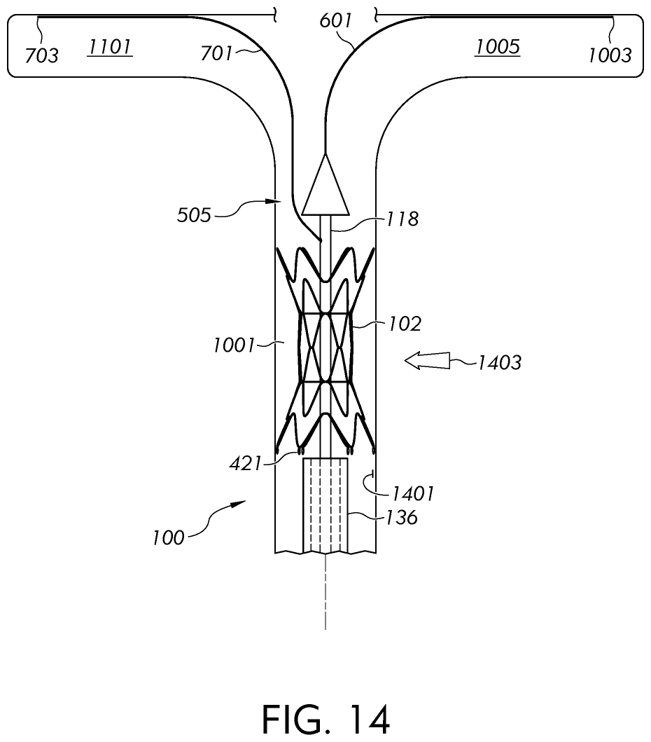

FIG. 4 illustrates further stages of deployment of the valve prosthesis 102 after the outer sheath 136 has been retracted proximally and the valve prosthesis 102 has been removed from the outer sheath 136 and allowed to self-expand. Once the heart valve prosthesis 102 is removed from the outer sheath 136, the holding catheter 128 can be remotely rotated from the proximal end 106, which can cause the coil 132 to likewise rotate. Rotation of the coil 132 can release the valve prosthesis 102 from the coil 132, for example as shown in FIG. 3, due to attachment members (e.g., loops 421 illustrated in FIGS. 4 and 14) at an end of the valve prosthesis 102 being released from the coil 132. Once the valve prosthesis 102 is released from the coil 132, the valve prosthesis 102 may expand to the radially-expanded position illustrated in FIG. 4. Portions of the implant apparatus 100, for example, the inner shaft member, that still extend through a lumen of the valve prosthesis 102 may be withdrawn and retracted, for example, by moving the implant apparatus 100 in the proximal direction 301.

As illustrated in FIG. 4, the valve prosthesis 102 can extend along a longitudinal axis 401 between an inflow end 403 and an outflow end 405, wherein the inflow end 403 is attached to the coil 132 in FIGS. 1-3. The valve prosthesis 102 can comprise a stent portion 407 that may move between the radially-expanded position and the radially-compressed position. In this way, the valve prosthesis 102 may be compressible to be inserted via catheter and expandable (e.g., self-expandable) to fit a desired body lumen, such as the right ventricular outflow tract (RVOT), for example. To further facilitate expansion of the valve prosthesis 102, in aspects, a balloon may be employed to cause the valve prosthesis 102 to move from the radially-compressed position to the radially-expanded position, for example, during a post-implant dilatation procedure. Although disclosed as self-expanding, the valve prosthesis 102 can be one or a combination of self-expandable, balloon-expandable, or mechanically expandable in other embodiments.

The stent portion 407 can comprise one or more materials that may be compressed but may regain a desired configuration upon release from compression. In aspects, the stent portion 407 can comprise a framework comprising a wire or plurality of wires 409 made of a shape-memory material (e.g., NITINOL™) and one or more pieces of fabric 411 (i.e., cloth, material, etc.) to which the wire or wires 409 can be attached. The wires 409 of the stent portion 407 can be shaped and aligned such that when the wires 409 are aligned generally coaxially, a central lumen can run along the length of the stent portion 407. In aspects, the wires 409 can comprise a series of sinusoidal bends around their circumference, which allow for the compression and expansion of the valve prosthesis 102 with minimal force. The wires 409 may be attached separately to the fabric 411. In addition, or alternatively, the wires 409 may be attached to each other at some or all of their adjacent apices. The valve prosthesis 102 comprises one or more valve leaflets disposed within and secured to the wires 409, with the valve leaflets configured to open and close to regulate blood flow through the valve prosthesis 102. The implant apparatus 100 and the valve prosthesis 102 are further described in U.S. Pat. Nos. 8,801,776 and 9,364,324, which are incorporated herein by reference.

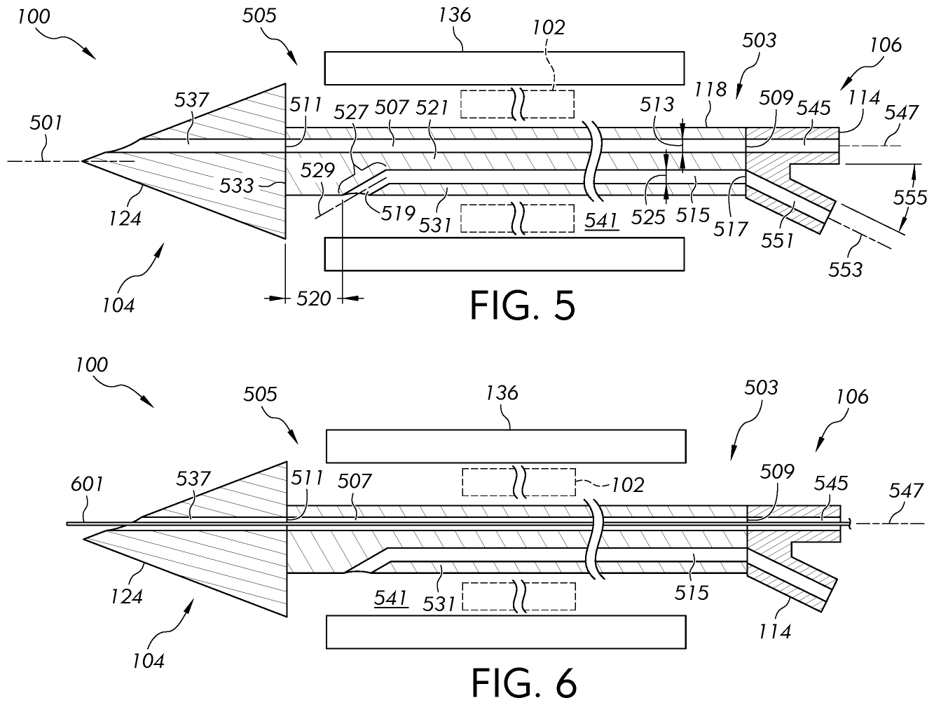

FIG. 5 illustrates a sectional view of portions of the implant apparatus 100 along lines 5-5 of FIG. 2, with the valve prosthesis 102 in the radially-compressed position and received within the outer sheath 136. It will be appreciated that portions of the implant apparatus 100 are illustrated schematically in FIGS. 5-14 for illustrative purposes. For example, the valve prosthesis 102 is illustrated somewhat schematically to show a position of the valve prosthesis 102 relative to the inner shaft member 118 and other portions of the implant apparatus 100. In operation, the outer sheath 136 may be coextensive with the valve prosthesis 102, such that when the outer sheath 136 is retracted, a distal end (e.g., outflow end 405) of the valve prosthesis 102 is no longer surrounded by the outer sheath 136, such that the distal end (e.g., outflow end 405) can release (e.g., partially deploy, radially expand, etc.). Accordingly, in aspects, the distal end of the valve prosthesis 102 can substantially match a location of the distal end of the outer sheath 136. The inner shaft member 118 can extend along a shaft axis 501 through the valve prosthesis 102 and through the outer sheath 136. It will be appreciated that the inner shaft member 118 can be bent, curved, flexed, or otherwise positioned in a non-linear orientation during movement through the patient's vasculature. The inner shaft member 118 can extend between a proximal shaft end 503 and a distal shaft end 505. The proximal shaft end 503 may be located near the proximal end 106 of the implant apparatus 100, and the distal shaft end 505 may be located near the distal end 104 of the implant apparatus 100. The inner shaft member 118 can comprise at least one lumen extending at least partially along a length of the inner shaft member 118 between the proximal shaft end 503 and the distal shaft end 505. For example, the inner shaft member 118 can comprise a first lumen 507 extending between a first entry opening 509 at the proximal shaft end 503 and a first exit opening 511 at the distal shaft end 505. In aspects, the first lumen 507 can comprise a first cross-sectional size, for example, a first diameter 513, that is within a range from about 0.5 millimeters to about 1.5 millimeters. However, based on a cross-sectional size of a guidewire that is received within the first lumen 507, which may be greater than 1.5 millimeters or less than 0.5 millimeters, the first diameter 513 of the first lumen 507 can be larger or smaller.

In addition, the inner shaft number 118 may comprise a second lumen 515 extending adjacent to the first lumen 507 between a second entry opening 517 at the proximal shaft end 503 and a second exit opening 519 spaced a distance 520 from the distal shaft end 505. A shaft wall 521 may be positioned between the first lumen 507 and the second lumen 515, with the shaft wall 521 extending partially or completely along the length of the inner shaft member 118 between the proximal shaft end 503 and the distal shaft end 505. In this way, the shaft wall 521 can separate the first lumen 507 from the second lumen 515, such that one guidewire can be maintained in the first lumen 507 and a separate guidewire can be maintained in the second lumen 515. In aspects, at least a portion of the first lumen 507 may be substantially parallel to at least a portion of the second lumen 515. The second lumen 515 can comprise a second cross-sectional size, for example, a second diameter 525, that is within a range from about 0.5 millimeters to about 1.5 millimeters. However, based on a cross-sectional size of a guidewire that is received within the second lumen 515, which may be greater than 1.5 millimeters or less than 0.5 millimeters, the second diameter 525 of the second lumen 515 can be larger or smaller. In aspects, the inner shaft member 118 can comprise a cross-sectional size (e.g., diameter) that is within a range from about 2 millimeters to about 4 millimeters, or about 2.5 millimeters to about 3.5 millimeters, or about 3 millimeters.

In this way, in some aspects, the first diameter 513 may be substantially equal to the second diameter 525. The second lumen 515 can comprise an end portion 527 that extends between the second exit opening 519 and the portion of the second lumen 515 that is parallel to the first lumen 507. In aspects, the end portion 527 can extend along a lumen axis 529 that is angled and non-parallel relative to the shaft axis 501 and angled and non-parallel to the first lumen 507. The end portion 527 can extend radially outwardly from the second lumen 515. In this way, the second exit opening 519 can be formed at, or can extend through, a circumferential sidewall 531 of the inner shaft member 118 while the first exit opening 511 can be formed at, or can extend through, an end wall 533 of the inner shaft member 118. The circumferential sidewall 531 may extend along the shaft axis 501 while the end wall 533 can be perpendicular to, and intersected by, the shaft axis 501.

The tip 124 can be attached to the distal shaft end 505 and may comprise a tip lumen 537 that is aligned with the first lumen 507. As used herein, by being aligned with, the first lumen 507 and the tip lumen 537 can be arranged end-to-end such that an axis can extend from the first lumen 507 and into the tip lumen 537 without intersecting a wall or other solid structure. In this way, a guidewire can extend from the first lumen 507 and into the tip lumen 537. The second exit opening 519 may be spaced apart from the tip 124, for example, by being spaced the distance 520 from the tip 124.

The outer sheath 136 can circumferentially surround the valve prosthesis 102 (e.g., when the valve prosthesis 102 is in the radially-compressed position) and the inner shaft member 118. The outer sheath 136 is movable relative to the valve prosthesis 102 and the inner shaft member 118. In aspects, the outer sheath 136 is movable relative to the inner shaft member 118 between a first delivery position (e.g., illustrated in FIGS. 2, 5, 6, and 10), in which the second exit opening 519 is within a sheath chamber 541 of the outer sheath 136, and a second delivery position (e.g., illustrated in FIGS. 3, 4, 7-9, and 11-14), in which the second exit opening 519 is outside of the sheath chamber 541. The sheath chamber 541 comprises the region that is circumferentially surrounded by the outer sheath 136. In this way, by being within the sheath chamber 541 in the first delivery position, the second exit opening 519 may be blocked by the outer sheath 136 such that a radial axis perpendicular to the shaft axis 501 can intersect the second exit opening 519 and the outer sheath 136. Conversely, by being outside of the sheath chamber 541 in the second delivery position, the second exit opening 519 is not blocked by the outer sheath 136 such that a radial axis perpendicular to the shaft axis 501 can intersect the second exit opening 519 but may not intersect the outer sheath 136.

The guide member 114 can be attached to the proximal shaft end 503 of the inner shaft member 118. In aspects, the guide member 114 can comprise a plurality of passageways that may be aligned with the first lumen 507 and the second lumen 515. For example, the guide member 114 can comprise a first passageway 545 that is aligned with the first entry opening 509 of the first lumen 507, with the first passageway 545 extending along a first guide axis 547 that can extend through the first entry opening 509. The guide member 114 can comprise a second passageway 551 that is aligned with the second entry opening 517 of the second lumen 515, with the second passageway 551 extending along a second guide axis 553 that can extend through the second entry opening 517. In aspects, the first guide axis 547 may be non-parallel relative to the second guide axis 553. For example, an angle 555 between the first guide axis 547 and the second guide axis 553 may be within a range from about 20 degrees to about 60 degrees. The guide member 114 can allow for a physician to insert one guidewire through the first passageway 545 and the first lumen 507, and insert a separate guidewire through the second passageway 551 and the second lumen 515. Further, the physician can hold the guide member 114 and control axial movement of the guidewires.

FIG. 6 illustrates the insertion of a first guidewire 601 within the inner shaft member 118. In this way, methods of implanting the heart valve prosthesis 102 can comprise positioning the first guidewire 601 within the first lumen 507 of the inner shaft member 118, with the first lumen 507 extending between the proximal shaft end 503 and the distal shaft end 505. For example, the first guidewire 601 can be positioned to extend through the first passageway 545 of the guide member 114, the first entry opening 509, the first lumen 507, the first exit opening 511, and the tip lumen 537. In this way, the first guidewire 601 can extend at least partially along the first guide axis 547. The first guidewire 601 can exit the implant apparatus 100 through the tip lumen 537 at the distal end 104. The first guidewire 601 comprises a cross-sectional size that is less than the first diameter 513 of the first lumen 507, such that the first guidewire 601 can move relative to the inner shaft member 118 and, likewise, the inner shaft member 118 can move relative to the first guidewire 601.

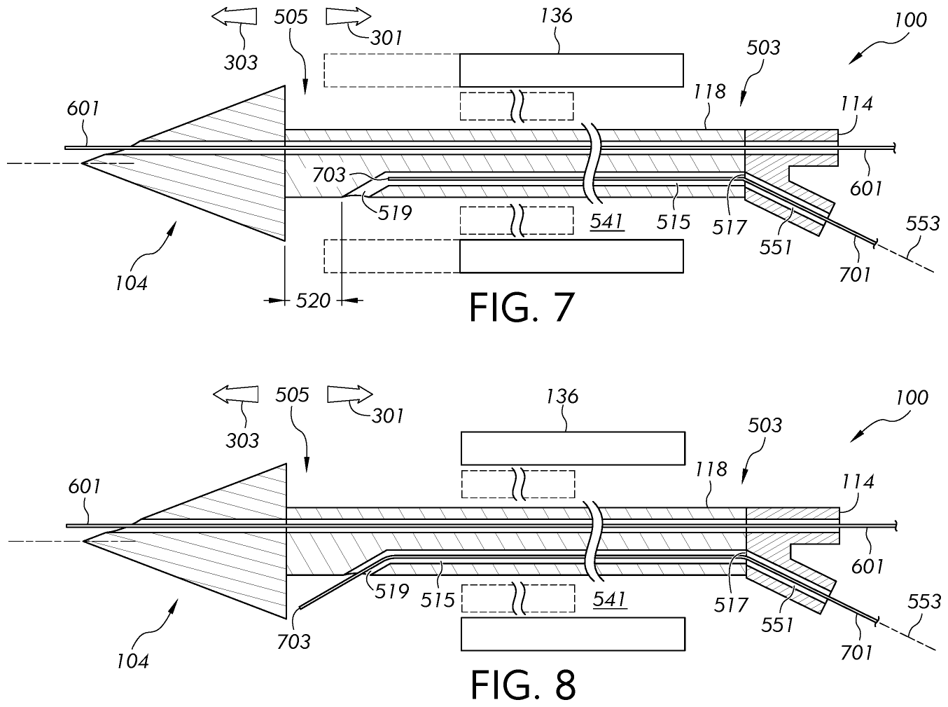

FIG. 7 illustrates the insertion of a second guidewire 701 within the inner shaft member 118. In this way, methods can comprise positioning the second guidewire 701 within the second lumen 515, with the second lumen 515 extending between the proximal shaft end 503 and the second exit opening 519 that is spaced the distance 520 from the distal shaft end 505. For example, the second guidewire 701 can extend through the second passageway 551, the second entry opening 517, and the second lumen 515. In this way, the portion of the second guidewire 701 extending through the second passageway 551 can extend along the second guide axis 553. In aspects, a second guidewire end 703 of the second guidewire 701 can remain within the second lumen 515 during movement of the implant apparatus 100. For example, methods can comprise moving (e.g., in the distal direction 303) the transcatheter heart valve implant apparatus 100 comprising the inner shaft member 118 and the valve prosthesis 102 along the first guidewire 601 toward a treatment site within a body lumen. During the movement of the transcatheter heart valve implant apparatus 100, the outer sheath 136 is in the first delivery position (e.g., illustrated in FIGS. 5, 6, and with dashed lines in FIG. 7) and can circumferentially surround the valve prosthesis 102 and the inner shaft member 118, with the second exit opening 519 located within the sheath chamber 541 of the outer sheath 136.

In aspects, upon reaching the treatment site, the outer sheath 136 can be moved in the proximal direction 301 and/or the inner shaft member 118 can be moved in the distal direction 303 such that the outer sheath 136 can be moved from the first delivery position to the second delivery position (e.g., illustrated with solid lines). In the second delivery position, the second exit opening 519 is not located within the sheath chamber 541. In this way, methods can comprise, prior to guiding the second guidewire 701 through the second exit opening 519, moving one or more of the outer sheath 136 relative to the inner shaft member 118 or the inner shaft member 118 relative to the outer sheath 136 such that the second exit opening 519 may be outside the sheath chamber 541.

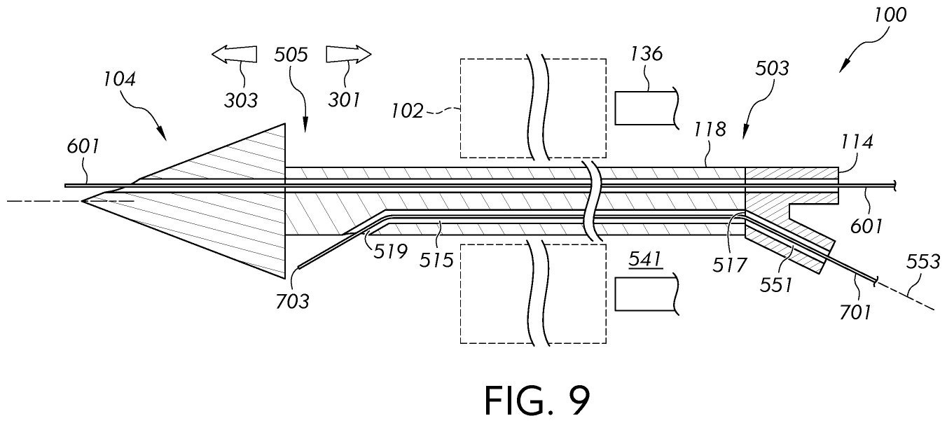

FIG. 8 illustrates the second guidewire 701 moving in the distal direction 303 relative to the inner shaft member 118 and passing through the second exit opening 519. The second guidewire 701 can be guided through the second exit opening 519 such that the second guidewire end 703 can be outside of the second lumen 515. To move the second guidewire 701, a physician can apply an axial force to the second guidewire 701 at the guide member 114 in the distal direction 303. This axial force can cause the second guidewire 701 to move through the second lumen 515 and exit the second lumen 515 through the second exit opening 519. Referring to FIG. 9, after the second guidewire 701 has exited the second exit opening 519, the valve prosthesis 102 can move from the radially-compressed position (e.g., illustrated in FIG. 8) to the radially-expanded position illustrated in FIG. 9. For example, the outer sheath 136 can be retracted, for example, by being moved in the proximal direction 301, such that the valve prosthesis 102 is not positioned in the sheath chamber 541. As described above relative to FIGS. 3-4, the coil 132 can be rotated to release the inflow end 403 of the valve prosthesis 102, thus allowing the valve prosthesis 102 to expand to the radially-expanded position.

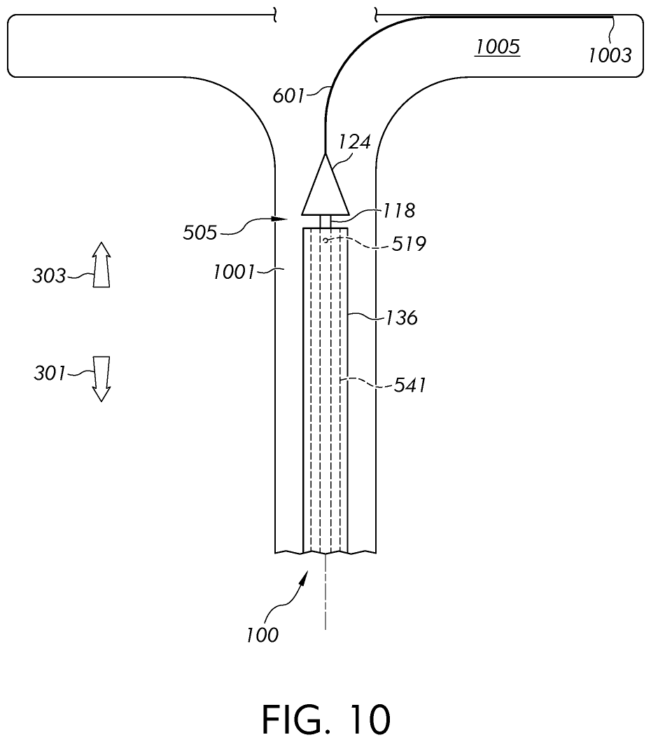

FIGS. 10-14 schematically illustrate a side view of the transcatheter heart valve implant apparatus 100 at a treatment site 1001. For example, FIG. 10 is a side view of the transcatheter heart valve implant apparatus 100 in a position similar to FIG. 6. In this way, a first guidewire end 1003 of the first guidewire 601 may be positioned at a first anchoring location 1005. In aspects, the first anchoring location 1005 can comprise a first pulmonary artery branch, such as the right pulmonary artery. Accordingly, methods can comprise guiding the first guidewire 601 through the first exit opening 511 (e.g., illustrated in FIG. 5) at the distal shaft end 505 such that the first guidewire end 1003 of the first guidewire 601 can be positioned in a first anchoring location, such as a first pulmonary artery branch. The implant apparatus 100 can move relative to the first guidewire 601, for example, in the distal direction 303, toward the treatment site 1001. In aspects, during the movement of the implant apparatus 100 in the distal direction 303, the second guidewire end 703 (e.g., illustrated in FIG. 7) can be located within the second lumen 515.

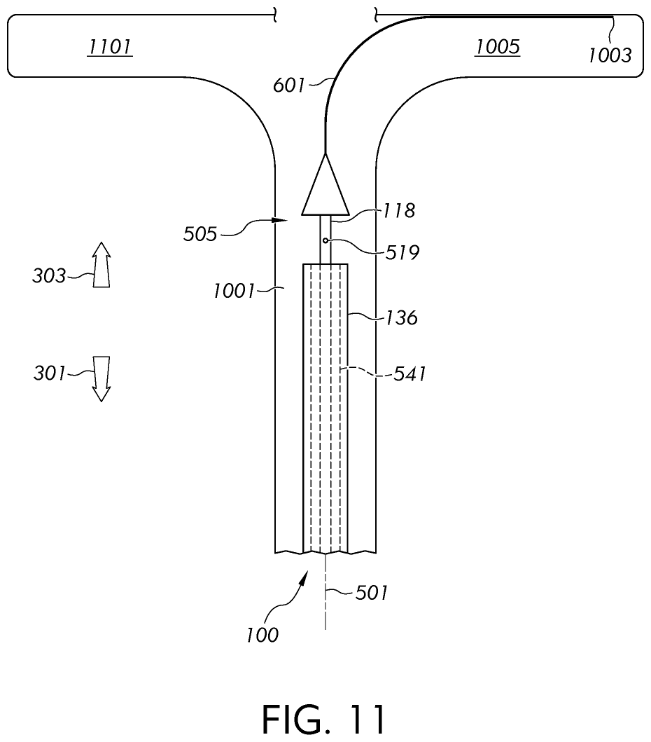

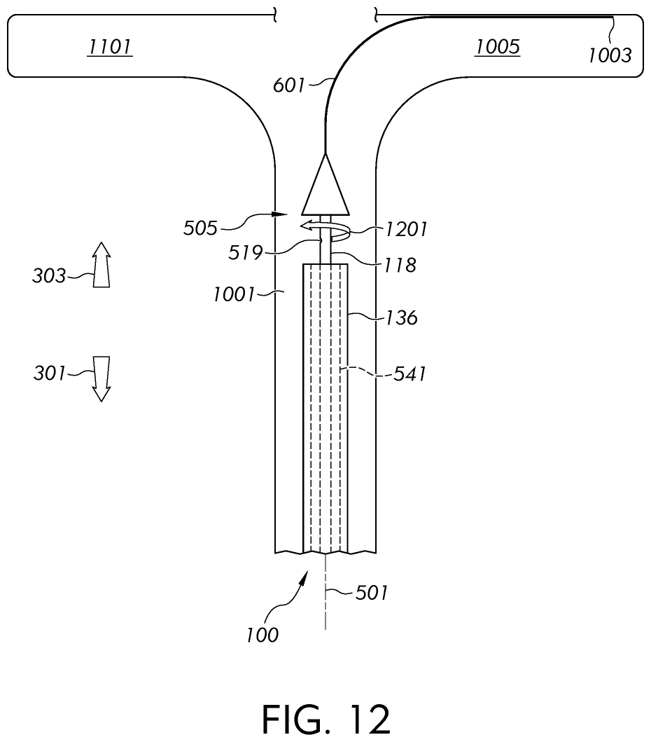

FIG. 11 illustrates a position of the transcatheter heart valve implant apparatus 100 substantially similar to FIG. 7. For example, the outer sheath 136 can be moved relative to the inner shaft member 118 and/or the inner shaft member 118 can be moved relative to the outer sheath 136. In this way, the second exit opening 519 may be outside of the sheath chamber 541 and no longer covered or blocked by the outer sheath 136. Referring to FIGS. 11-12, in aspects, prior to guiding the second guidewire 701 through the second exit opening 519, methods can comprise rotating the inner shaft member 118 relative to the outer sheath 136 such that the second exit opening 519 may be oriented to face a second anchoring location 1101. The inner shaft member 118 can be rotated about the shaft axis 501. The second anchoring location 1101 can comprise, for example, a second anchoring location, such as, for example, a second pulmonary artery branch (e.g., the left pulmonary artery). For example, the inner shaft member 118 can be rotatable relative to the outer sheath 136 about the shaft axis 501 such that in a first rotational position (e.g., as illustrated in FIG. 11), the second exit opening 519 faces a first location of the outer sheath 136 and may not face the second anchoring location 1101. The inner shaft member 118 can be rotated (e.g., rotation illustrated schematically with arrowhead 1201 about the shaft axis 501 in FIG. 12) from the first rotational position of FIG. 11 to a second rotational position of FIG. 12, in which the second exit opening 519 faces a second location of the outer sheath 136 and faces the second anchoring location 1101. In this way, the rotation 1201 can occur while the second exit opening 519 is outside of the sheath chamber 541. The inner shaft member 118 can be rotated in a clockwise direction or a counter-clockwise direction, with the end result being that the second exit opening 519 faces the second anchoring location 1101. In aspects, by facing the second anchoring location 1101, an axis can extend from the second exit opening 519 and into the second anchoring location 1101 without intersecting any structures, such as native anatomical structures of the patient and/or components of the implant apparatus 100. Accordingly, in this way, when the inner shaft member 118 is in the second rotational position of FIG. 12, an unobstructed path may exist between the second exit opening 519 and the second anchoring location 1101.

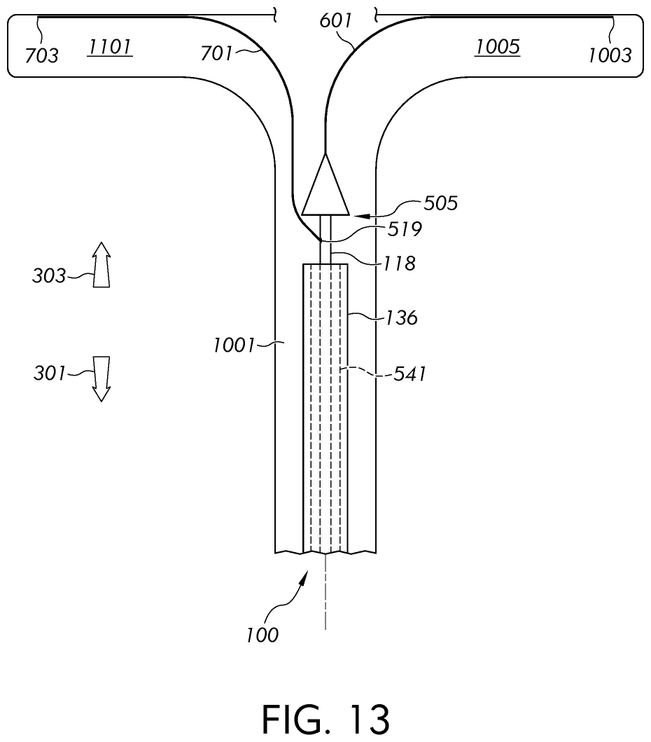

FIG. 13 illustrates a position of the transcatheter heart valve implant apparatus 100 substantially similar to FIG. 8. For example, the second guidewire 701 can be guided through the second exit opening 519 such that the second guidewire end 703 can be positioned in the second anchoring location 1101, for example, the second pulmonary artery branch. By guiding the second guidewire 701 through the second exit opening 519, the second guidewire 701 can be moved in the distal direction 303 such that the second guidewire end 703 moves toward the second anchoring location 1101. The second guidewire 701 can continue to be moved in the distal direction 303 at least until the second guidewire end 703 is located in the second anchoring location 1101, for example, by contacting a wall of the second pulmonary artery branch. As described above, the first guidewire 601 and the second guidewire 701 can be moved in the distal direction 303 due to a force being applied to the guidewires 601, 701 at the guide member 114 (e.g., illustrated in FIGS. 5-9).

In this way, the first guidewire 601 and the second guidewire 701 may be simultaneously deployed prior to the deployment of the valve prosthesis 102, with the first guidewire 601 in the first anchoring location 1005 and the second guidewire 701 in the second anchoring location 1101. In aspects, the first guidewire 601, for example, the first guidewire end 1003, can be in contact with a wall of the first anchoring location 1005. Likewise, the second guidewire 701, for example, the second guidewire end 703, can be in contact with a wall of the second anchoring location 1101. In aspects, the first guidewire 601 and the second guidewire 701 can be spaced apart, for example, within a range from about 135 degrees to about 225 degrees, or from about 160 degrees to about 200 degrees. Together, the first guidewire 601 and the second guidewire 701 can function to provide opposing forces to the inner shaft member 118 and limit unintended movement of the implant apparatus 100 during deployment of the valve prosthesis 102. In aspects, the anchoring locations 1005, 1101 for the first guidewire 601 and the second guidewire 701 can be switched. For example, the first guidewire 601 can be positioned in the second anchoring location 1101 (e.g., the left pulmonary artery), and the second guidewire 701 can be positioned in the first anchoring location 1005 (e.g., the right pulmonary artery). Despite the guidewires 601, 701 being capable of positioning in either of the anchoring locations 1005, 1101, the guidewires 601, 701 can be simultaneously deployed. As used herein, the term simultaneous (e.g., simultaneous deployment, simultaneous positioning, etc.) can refer to the first guidewire 601 and the second guidewire 701 both extending, at the same time, to an exterior of the inner shaft member 118 and extending into differing anchoring locations. In this way, at a given moment, the first guidewire 601 may extend into the first anchoring location 1005 at the same time as the second guidewire 701 extending into the second anchoring location 1101.

FIG. 14 illustrates the deployment of the valve prosthesis 102 in a similar manner as described relative to FIG. 9. As the valve prosthesis 102 is deployed, the first guidewire 601 and the second guidewire 701 can anchor the inner shaft member 118 to ensure that the valve prosthesis 102 is deployed at a desired location. For example, during deployment, the valve prosthesis 102 may be in proximity to a wall 1401 of the treatment site 1001. As the valve prosthesis 102 radially expands from the radially-contracted position (e.g., illustrated in FIG. 13) to the radially-expanded position (e.g., illustrated in FIG. 14), the valve prosthesis 102 may contact the wall 1401. Contact between the valve prosthesis 102 and the wall 1401 may generate a force 1403 (e.g., illustrated schematically with arrowhead) acting upon the valve prosthesis 102 in a direction away from the wall 1401. In aspects, this force 1403 can tend to move the valve prosthesis 102 and cause an inaccurate positioning of the valve prosthesis 102 within the treatment site 1001.

The simultaneous anchoring and positioning of the first guidewire 601 in the first anchoring location 1005 and the second guidewire 701 in the second anchoring location 1101 can counteract the force 1403 and limit unintended movement of the valve prosthesis 102. For example, the simultaneous anchoring of the first guidewire 601 and the second guidewire 701 can provide a balanced reaction force on the inner shaft member 118, such that the inner shaft member 118 can remain centered as the valve prosthesis 102 is deployed and even if the valve prosthesis 102 contacts the wall 1401. That is, in aspects, the first anchoring location 1005 and the second anchoring location 1101 are angularly spaced apart, with the guidewires 601, 701 in contact with walls of the anchoring locations 1005, 1101. The guidewires 601, 701 can therefore assist in resisting movement of the inner shaft member 118 and the valve prosthesis 102 despite the force 1403 possibly acting upon the valve prosthesis 102. Accordingly, the valve prosthesis 102 can be positioned at a desired location at the treatment site 1001 with a reduced likelihood of misplacement. Following the deployment of the valve prosthesis 102, the guidewires 601, 701 can be retracted in the proximal direction 301 and the implant apparatus 100 can be removed from the vasculature of the patient. The implant apparatus 100 described herein can provide several benefits, such as, for example, more accurate positioning of the valve prosthesis 102. In addition, the processes and structures related to the deployment of the valve prosthesis 102 need not be changed. That is, the holding catheter 128, the coil 132, etc. can function in a similar manner as existing techniques.

It should be understood that while various aspects have been described in detail relative to certain illustrative and specific examples thereof, the present disclosure should not be considered limited to such, as numerous modifications and combinations of the disclosed features are possible without departing from the scope of the following claims.

What is claimed is:

1. A transcatheter heart valve implant apparatus comprising:
an annular valve prosthesis extending along a longitudinal axis between an inflow end of the valve prosthesis and an outflow end of the valve prosthesis;
an inner shaft member extending along a shaft axis through the valve prosthesis between a proximal shaft end and a distal shaft end, the inner shaft member comprising:
a first lumen extending between the proximal shaft end and a first exit opening at the distal shaft end; and
a second lumen extending adjacent to the first lumen between the proximal shaft end and a second exit opening spaced a distance from the distal shaft end, a shaft wall positioned between the first lumen and the second lumen;
a first guidewire extending through the first lumen and the first exit opening, wherein a first end of the first guidewire is configured to be positioned at a first anchoring location; and
a second guidewire extending through the second lumen and the second exit opening, wherein a second end of the second guidewire is configured to be positioned at a second anchoring location simultaneously with the first guidewire positioned at the first anchoring location.

2. The transcatheter heart valve implant apparatus of claim 1, further comprising a tip attached to the distal shaft end, the tip comprising a tip lumen that is aligned with the first lumen such that the first guidewire extends through the tip lumen.

3. The transcatheter heart valve implant apparatus of claim 2, wherein the second exit opening is spaced apart from the tip.

4. The transcatheter heart valve implant apparatus of claim 1, wherein the first anchoring location comprises a first pulmonary artery branch and the second anchoring location comprises a second pulmonary artery branch.

5. The transcatheter heart valve implant apparatus of claim 1, further comprising an outer sheath circumferentially surrounding the valve prosthesis and the inner shaft member, the outer sheath movable relative to the inner shaft member such that in a first delivery position, the second exit opening is within a sheath chamber of the outer sheath, and in a second delivery position, the second exit opening is outside of the sheath chamber.

6. The transcatheter heart valve implant apparatus of claim 5, wherein the inner shaft member is configured to be rotatable relative to the outer sheath such that in a first rotational position, the second exit opening faces a first location of the outer sheath, and in a second rotational position, the second exit opening faces a second location of the outer sheath.

7. The transcatheter heart valve implant apparatus of claim 1, wherein the first lumen comprises a first diameter that is within a range from about 0.5 millimeters to about 1.5 millimeters, and the second lumen comprises a second diameter that is within a range from about 0.5 millimeters to about 1.5 millimeters.

8. The transcatheter heart valve implant apparatus of claim 1, wherein the first lumen is substantially parallel to the second lumen.

9. A transcatheter heart valve implant apparatus comprising:
an annular valve prosthesis extending along a longitudinal axis between an inflow end of the valve prosthesis and an outflow end of the valve prosthesis;
an inner shaft member extending along a shaft axis through the valve prosthesis between a proximal shaft end and a distal shaft end, the inner shaft member comprising:
a first lumen extending between a first entry opening at the proximal shaft end and a first exit opening at the distal shaft end; and
a second lumen extending adjacent to the first lumen between a second entry opening at the proximal shaft end and a second exit opening spaced a distance from the distal shaft end, a shaft wall positioned between the first lumen and the second lumen;
a first guidewire extending through the first entry opening, the first lumen, and the first exit opening, wherein a first end of the first guidewire is configured to be positioned in a first pulmonary artery branch; and
a second guidewire extending through the second entry opening, the second lumen, and the second exit opening, wherein a second end of the second guidewire is configured to be positioned in a second pulmonary artery branch simultaneously with the first guidewire positioned in the first pulmonary artery branch.

10. The transcatheter heart valve implant apparatus of claim 9, further comprising a guide member attached to the proximal shaft end of the inner shaft member.

11. The transcatheter heart valve implant apparatus of claim 10, wherein the guide member comprises:
a first passageway that is aligned with the first entry opening of the first lumen such that the first guidewire extends through the first passageway, the first entry opening, and the first lumen, the first passageway extending along a first guide axis; and
a second passageway that is aligned with the second entry opening of the second lumen such that the second guidewire extends through the second passageway, the second entry opening, and the second lumen, the second passageway extending along a second guide axis.

12. The transcatheter heart valve implant apparatus of claim 11, wherein an angle between the first guide axis and the second guide axis is within a range from about 20 degrees to about 60 degrees.

13. The transcatheter heart valve implant apparatus of claim 9, further comprising a tip attached to the distal shaft end, the tip comprising a tip lumen that is aligned with the first lumen such that the first guidewire extends through the tip lumen.

14. The transcatheter heart valve implant apparatus of claim 13, wherein the second exit opening is spaced apart from the tip.

15. A method of implanting a valve prosthesis comprising:
positioning a first guidewire within a first lumen of an inner shaft member, the first lumen extending between a proximal shaft end of the inner shaft member and a distal shaft end of the inner shaft member;
positioning a second guidewire within a second lumen of the inner shaft member, the second lumen extending between the proximal shaft end and a second exit opening spaced a distance from the distal shaft end;
guiding the first guidewire through a first exit opening at the distal shaft end such that a first guidewire end of the first guidewire is positioned in a first anchoring location;
moving a transcatheter heart valve implant apparatus comprising the inner shaft member and the valve prosthesis along the first guidewire toward a treatment site within a body lumen;
guiding the second guidewire through the second exit opening such that a second guidewire end of the second guidewire is positioned in a second anchoring location; and
deploying the valve prosthesis at the treatment site while the first guidewire end is positioned in the first anchoring location and the second guidewire end is positioned in the second anchoring location.

16. The method of claim 15, wherein during the moving of the transcatheter heart valve implant apparatus, an outer sheath circumferentially surrounds the valve prosthesis and the inner shaft member, and the second exit opening is within a sheath chamber of the outer sheath.

17. The method of claim 16, further comprising, prior to guiding the second guidewire through the second exit opening, moving one or more of the outer sheath relative to the inner shaft member or the inner shaft member relative to the outer sheath such that the second exit opening is outside the sheath chamber.

18. The method of claim 17, further comprising, prior to guiding the second guidewire through the second exit opening, rotating the inner shaft member relative to the outer sheath such that the second exit opening is oriented facing the second anchoring location.

19. The method of claim 18, wherein the rotating occurs while the second exit opening is outside the sheath chamber.

20. The method of claim 15, wherein during the moving of the transcatheter heart valve implant apparatus, the second guidewire end is located within the second lumen, and wherein the first anchoring location comprises a first pulmonary artery branch and the second anchoring location comprises a second pulmonary artery branch.
